(19) United States
(12) Patent Application Publication
(10) Pub. No.: US 2017/0224556 A1
(43) Pub. Date: Aug. 10, 2017

(54) POUCH FOR AN ABSORBENT HYGIENIC ARTICLE WHICH COMPRISES A TRANSVERSAL OPENING INDICATION

(71) Applicant: ALBAAD MASSUOT YITZHAK LTD., Massuot Yitzhak (IL)

(72) Inventor: Lois GUZMAN, Lexington, KY (US)

(21) Appl. No.: 15/040,712

(22) Filed: Feb. 10, 2016

(51) Int. Cl. A61F 13/551; B65D 75/66; B65D 75/04

(52) U.S. Cl. CPC A61F 13/5514; B65D 75/04; B65D 75/66

(57) ABSTRACT

A pouch for an absorbent hygienic article. The pouch has: (a) a plastic film sheet; (b) a transversal opening edge at a folded plastic film sheet; (c) a transversal strip which is printed on the external surface of said plastic film sheet adjacent and in parallel to said opening edge, said printed strip having a first printed pattern; and (d) a decoration applied to the rest of the external surface of said plastic film sheet. The decoration has a second pattern which is different from said first pattern of the strip. The transversal printed strip provides a distinctive identification to a consumer with respect to the location of the opening edge of the pouch, indicating (i) a finger-lift location; (ii) a finger-grip location; and (iii) is used as a reference to wrap a used disposable pad.

### FIELD OF INVENTION

The invention relates in general to the field of absorbent articles for hygienic use. More specifically, the invention relates to a pouch for an absorbent article which includes a transversal indication to guide the user where to open the article.

### BACKGROUND OF THE INVENTION

Absorbent articles for hygienic use are in many cases sold in a folded form, while a plurality, for example, 10-20 articles are sold within one secondary packing. Typically, for hygienic purposes, each of said articles is also enveloped within a film wrap, hereinafter also referred to as “pouch”. The cover which forms the pouch is typically made of a rectangular sheet of film (typically made of plastic, paper, non-woven material, etc.—hereinafter referred to as “plastic film”) which is folded about two transversal folding axes, and thermally sealed at its two longitudinal margins to form a closed envelope in which the article is positioned at a hygienic condition (the terms “longitudinal” and “transversal” are used herein for the sake of convenience and better clarity, and they are defined herein based on the typical manner seen to the user just before opening of the pouch). In some cases, the thin sheet of typically plastic film is glued (before folding) to an external surface of the article, and both the plastic film cover and the article are folded together. In said latter case, the plastic film cover in fact serves two purposes: (a) it keeps the hygiene of the article when kept in its packaged form; and (b) it isolates the user's hand from the user's body and body fluids during the positioning of the article for use.

As noted, at each of the two longitudinal margins of the plastic film pouch the respective opposing surfaces of the plastic film cover are glued one another. One transversal edge of the folded plastic film sheet is typically used for opening the pouch. In some cases, said transversal edge is also glued to an opposing surface of the plastic film cover that faces this edge to form a fully closed envelope, and in other cases, this edge is unglued, while the pouch is kept in a essentially closed arrangement by said glued longitudinal margins. In any case, and in order to use the hygienic article, the user has to locate said transversal edge of the plastic film sheet, and to pull it in order to un-tear the glued margins.

The thin plastic film cover is typically colored, and sometimes also decorated. In such a situation, the opening transversal edge (whether glued or not) becomes somewhat hidden or hardly seen, particularly as there are top and bottom surfaces for the substantially flat pouch.

In order to make the transversal edge more visible, one prior art pouch, manufactured by Always is provided with a longitudinal tape which is glued on top of the transversal edge, substantially at equal distances between the two longitudinal margins. Although this tape improves the visibility of the transversal edge, still this arrangement requires an additional element which is separate from the plastic film cover. Some other pouches lack any of such visible indication.

It is therefore an object of the invention to provide an opening indication within the pouch, which is much more visible compared to the prior art.

It is another object of the invention to provide an opening indication within the pouch which does not require any additional element, beyond the plastic film cover.

It is still another object of the invention to provide an opening indication which is in fact an integral part of the plastic film cover.

It is still another object of the invention to provide said transversal opening indication in an easy and low cost manner.

Other advantages of the invention will become apparent as the description proceeds.

### SUMMARY OF THE INVENTION

The invention relates to a pouch for an absorbent hygienic article comprising: (a) a plastic film sheet which is folded to envelope said absorbent hygienic article; (b) a transversal opening edge at said folded plastic film sheet; (c) a transversal strip which is printed on the external surface of said plastic film sheet adjacent and in parallel to said opening edge, said printed strip having a first printed pattern; and (d) a decoration which is applied to the rest of the external surface of said plastic film sheet, said decoration having a second pattern which is different from said first pattern of the strip; wherein said transversal printed strip provides a distinctive identification to a consumer with respect to the location of the opening edge of the pouch, indicating (i) a finger-lift location to open the pouch; (ii) a finger-grip location to release the pouch from the article after positioning it in place; and (iii) is used as a reference to wrap a used disposable pad.

In an embodiment of the invention, the pouch further comprises two longitudinal side margins, each of said margins being formed by bonding two opposite surfaces of said folded plastic film sheet one to the other.

In an embodiment of the invention, the pouch is formed by folding said plastic film sheet about two transversal axes.

### BRIEF DESCRIPTION OF THE DRAWINGS

FIG. 1 shows a bottom view of a pouch for a hygienic absorbent article according to an embodiment of the invention;

FIG. 2 shows a top view the pouch of FIG. 1;

FIG. 3 is a perspective view of the pouch of FIGS. 1 and 2; and

FIG. 4 shows a perspective view of a pouch according to another embodiment.

### DETAILED DESCRIPTION OF PREFERRED EMBODIMENTS

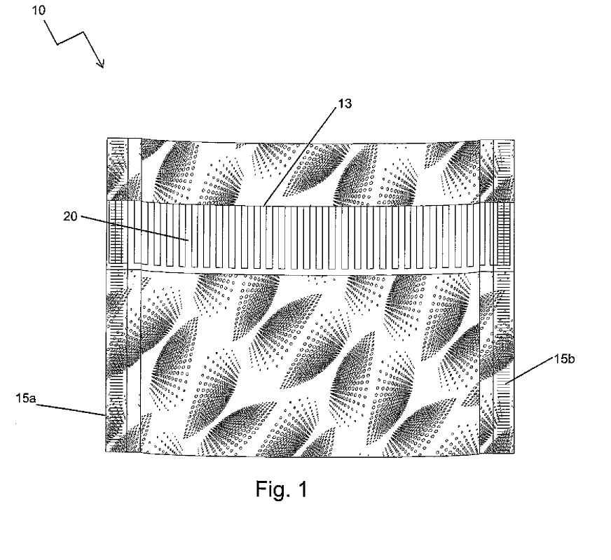

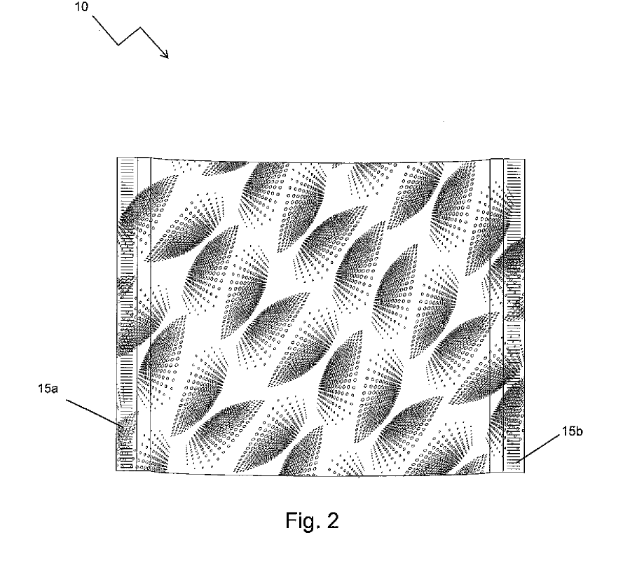

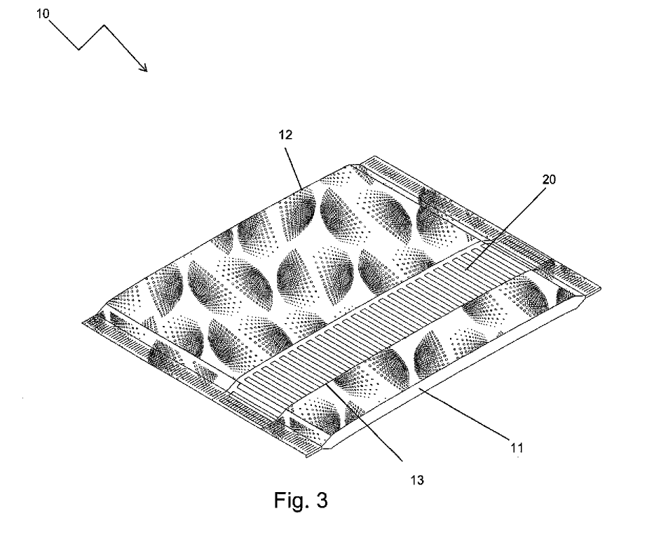

FIG. 3 shows in a perspective view a pouch 10 for a hygienic article, according to the present invention. FIGS. 1 and 2 show bottom and top views, respectively, of the pouch of FIG. 1.

As noted above, pouch 10 is made of a thin, substantially rectangular plastic (or paper, or nonwoven material) film sheet, which is folded twice about two axes 11, and 12, such that a first transversal edge of the rectangular sheet is hidden internally, and the second transversal edge is indicated by numeral 13. The folded plastic film sheet is glued at the two longitudinal margins, 15a, and 15b, respectively, and in such a manner the hygienic article is in fact enveloped by the plastic film sheet.

In one typical case, the nylon sheet is glued at transversal edge 13 to the plastic film below. In another typical case, transversal edge 13 remains unglued, while the glued margins 15a and 15b are sufficient for maintaining the enveloping of the article (not shown).

The enveloping plastic film is typically colored, and in some cases also has some form of decoration, as shown in the example of FIGS. 1, 2, and 3.

All the elements of the pouch 10 that have been described so far are typical to the pouches of the prior art. In order to open the pouch, and to bring the article into use, the user has to hold the pouch by one hand, to pull the transversal edge 13, thereby to tear the glued margins 15a and 15b, and thereby to expose the previously enveloped article. However, the edge 13 is typically not easily visible in the prior art pouches, particularly as the bottom and top sides have substantially a same color and/or a same decoration pattern, and due to the fact that a thin plastic film sheet is used.

As noted above, in order to improve visibility, the Always brand provides a longitudinal tape (for example white) which is glued to the nylon in proximity to the edge 13. However, this involves the complication of using an additional element. In some other cases, there are no specific indications for the location of the opening transversal edge 13.

In order to improve the visibility of the transversal edge 13, and according to the present invention, a transversal strip 20 is provided in parallel and adjacent the edge 13. Strip 13 is in fact printed on the plastic film sheet, in a different color and/or pattern than the pattern and color of the rest of the plastic sheet, for example as shown in FIGS. 1-3. In such a manner, the visibility of the edge 13 is significantly improved, and the user can easily find the location of the opening edge of the pouch. Moreover the applying of strip 20 onto the plastic film is very simple, as it only involves modification of the pattern or decoration which is in any case typically applied to the plastic film sheet. This is in contrast, for example, to the arrangement as used in the pouch of Always.

The transversal printed strip provides a distinctive identification to the consumer with respect to the location of the edge of the transversal part of the film, indicating (a) a finger-lift location to open the pouch; (b) a finger-grip location to release the pouch from the product after positioning it in place; and (c) is used as a reference to wrap the used disposable pad.

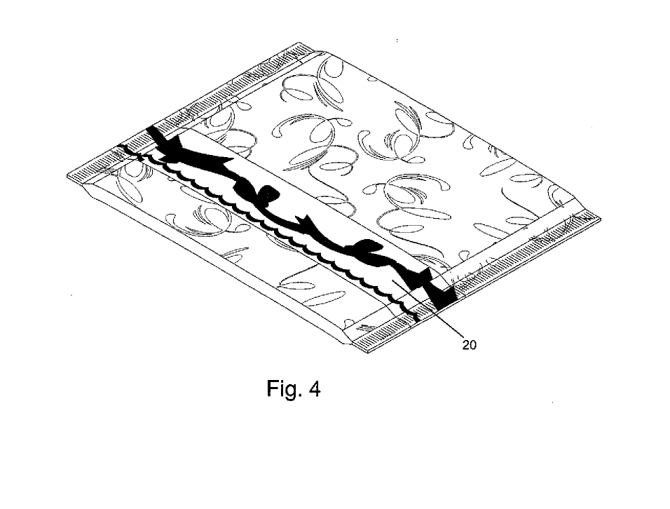

Any pattern, decoration, or color may be applied within strip 20, as long as it is different, respectively from the pattern, decoration, or color on the rest of the nylon sheet. FIG. 4 shows still another exemplary pattern for use within the strip 20.

While some embodiments of the invention have been described by way of illustration, it will be apparent that the invention can be carried into practice with many modifications, variations and adaptations, and with the use of numerous equivalents or alternative solutions that are within the scope of persons skilled in the art, without departing from the spirit of the invention or exceeding the scope of the claims.

1. A pouch for an absorbent hygienic article comprising:
a. a plastic film sheet which is folded to envelope said absorbent hygienic article;
b. a transversal opening edge at said folded plastic film sheet;
c. a transversal strip which is printed on the external surface of said plastic film sheet adjacent and in parallel to said opening edge, said printed strip having a first printed pattern; and
d. a decoration which is applied to the rest of the external surface of said plastic film sheet, said decoration having a second pattern which is different from said first pattern of the strip;
wherein said transversal printed strip provides a distinctive identification to a consumer with respect to the location of the opening edge of the pouch, indicating (i) a finger-lift location to open the pouch; (ii) a finger-grip location to release the pouch from the article after positioning it in place; and (iii) is used as a reference to wrap a used disposable pad.

2. A pouch according to claim 1, further comprising two longitudinal side margins, each of said margins being formed by bonding two opposite surfaces of said folded plastic film sheet one to the other.

3. A pouch according to claim 1, wherein the pouch is formed by folding said plastic film sheet about two transversal axes.
